(19) United States
(12) Patent Application Publication
(10) Pub. No.: US 2023/0329806 A1
(43) Pub. Date: Oct. 19, 2023

(54) SURGICAL DECISION SUPPORT SYSTEM BASED ON AUGMENTED REALITY (AR) AND METHOD THEREOF

(71) Applicant: SMART SURGERY, Taipei City (TW)

(72) Inventors: Yu-Chieh LEE, Taipei City (TW); Yi-Ta SHEN, Taipei City (TW); Hsin-Man CHIANG, Taipei City (TW)

(21) Appl. No.: 18/132,368

(22) Filed: Apr. 8, 2023

Related U.S. Application Data
(60) Provisional application No. 63/330,791, filed on Apr. 14, 2022.

(51) Int. Cl. A61B 34/00; G16H 20/40

(52) U.S. Cl. CPC A61B 34/25; G16H 20/40; A61B 2034/105

(57) ABSTRACT

A surgical decision support system based on augmented reality (AR) and a method thereof are disclosed. In the surgical decision support system, a surgeon can create an optimal surgical operation before a surgical operation; during a surgical operation, the optimal surgical operation can be demonstrated through augmented reality, a current surgical operation is detected and compared with the optimal surgical operation, a difference message is displayed to provide a surgical decision support when a comparison difference exceeds a tolerable range. Therefore, the technical effect of improving operation efficiency and success rate can be achieved.

### CROSS REFERENCE STATEMENT

The present application is based on, and claims priority from, U.S. Provisional Patent Application Ser. No. 63/330,791, filed Apr. 14, 2022, the disclosure of which is hereby incorporated by reference herein in its entirety.

### BACKGROUND

### 1. Technical Field

The present invention relates to a surgical decision system and a method thereof, and more particularly to a surgical decision support system based on augmented reality and a method thereof.

### 2. DESCRIPTION OF THE RELATED ART

In recent years, with the popularity and vigorous development of augmented reality, various applications based on augmented reality has sprung up, for example, the augmented reality is generally applied in surgery, navigation and games.

Generally speaking, the conventional method of applying augmented reality in a surgical operation is to create virtual-real environment through a camera device and a display device; in the environment, medical data, images, and organ models are displayed as virtual objects on the display device, a real image captured by the camera device is displayed on the display device at the same time, so that a surgeon can see the virtual objects and the real image at the same time to receive a surgical reference when browsing the display device. However, in the actual operation process, the above-mentioned conventional method just passively provides information and is unable to actively provide corresponding surgical decision support according to the surgeon's surgical operation. In other words, when a surgeon is inexperienced, the above-mentioned conventional method just provides the support with a large amount of relevant data and images in real time, the inexperienced surgeon may be unable to get help from the support, and be distracted and have more pressure to led to surgical errors. Therefore, so above-mentioned conventional method has a problem that surgeons may make mistakes due to inexperience or pressure.

Some manufacturers proposed to a method of using augmented reality to simulate organs and providing surgical demonstration operations to teach surgeons. In the proposed method, organ models are created as virtual objects in advance, and surgical demonstration operations are displayed for surgeons to learn, thereby indirectly improving the experience of surgeons. However, there is a huge gap between the actual operation process and the learning process, that is, even if studying and practicing many times in advance, an inexperienced surgeon usually still may feel huge pressure during the actual operation, especially when an unexpected situation occurs during the operation process. Therefore, the above-mentioned conventional methods are unable to effectively solve the problem that surgeons may make mistakes due to inexperience or pressure.

According to above-mentioned contents, what is needed is to develop an improved solution to solve the problem that surgeons may make mistakes due to inexperience or pressure.

### SUMMARY

An objective of the present invention is to disclose a surgical decision support system based on augmented reality and a method thereof, to solve the above-mentioned conventional problem.

In order to achieve the objective, the present invention discloses a surgical decision support system based on augmented reality, the surgical decision support system includes a surgical database and a host. The surgical database is configured to store surgical plans, wherein each of the surgical plans comprises an organ model, an operational process, a time point of using surgical instrument, and physiological data, each of the surgical plans is presentable through augmented reality.

The host is linked with the surgical database, and includes a non-transitory computer-readable storage medium and a hardware processor. The non-transitory computer-readable storage medium is configured to store computer-readable program instructions. The hardware processor is electrically connected to the non-transitory computer-readable storage medium, and configured to execute the computer-readable program instructions to execute a training module, a sensing module, and a decision support module. The training module is linked with the surgical database, wherein before a surgery is performed, the training module is configured to select and load a corresponding one of the surgical plans for training, present the loaded surgical plan in augmented reality, enable sensors to continuously sense a free motion of a surgical instrument in a three-dimensional space during training, so as to create an optimal surgical operation. During a process of performing the surgery, the sensing module is configured to enable the sensors to continuously sense the free motion of the surgical instrument in three-dimensional space to generate a current surgical operation. The decision support module is connected to the training module and the sensing module, configured to compare the optimal surgical operation and the current surgical operation, wherein when a comparison difference exceeds a tolerable range, the decision support module outputs a difference message to provide a surgical decision support.

In order to achieve the objective, the present invention discloses a surgical decision support method based on augmented reality, the surgical decision support method is executed by a host linked with a surgical database, and includes steps of: storing surgical plans in the surgical database, wherein each of the surgical plan comprises an organ model, an operational process, a time point of using surgical instrument, and physiological data, and each of the surgical plan is presentable through augmented reality; before a surgery is performed, selecting and loading one of the surgical plans that corresponds to the surgery for training, and presenting the loaded surgical plan through augmented reality, enabling sensors to continuously sense a free motion of a surgical instrument in a three-dimensional space to create an optimal surgical operation during the training, by the host; during a process of performing the surgery, enabling the sensors to continuously sense the free motion of the surgical instrument to generate a current surgical operation, by the host; comparing the optimal surgical operation and the current surgical operation, and outputting a difference message to provide a surgical decision support when a comparison difference exceeds a tolerable range, by the host.

According to the above-mentioned system and the method of the present invention, the difference between the present invention and conventional technology is that, in the present invention, a surgeon can create the optimal surgical operation before a surgical operation; during the surgical operation, the optimal surgical operation can be demonstrated through augmented reality, the current surgical operation is detected and compared with the optimal surgical operation, the difference message is displayed to provide the surgical decision support when the comparison difference exceeds the tolerable range.

Therefore, the technical solution of the present invention is able to achieve the technical effect of improving operation efficiency and success rate.

### BRIEF DESCRIPTION OF THE DRAWINGS

The structure, operating principle and effects of the present invention will be described in detail by way of various embodiments which are illustrated in the accompanying drawings.

FIG. 1 is a system block diagram of a surgical decision support system based on augmented reality, according to the present invention.

FIG. 2A to FIG. 2C are flowcharts of a surgical decision support method based on augmented reality, according to the present invention.

FIG. 3A and FIG. 3B are schematic views of providing decision supports in a surgical operation at different time points, according to an application of the present invention.

FIG. 4 is a schematic view of setting a response plan, according to the present invention.

### DETAILED DESCRIPTION

The following embodiments of the present invention are herein described in detail with reference to the accompanying drawings. These drawings show specific examples of the embodiments of the present invention. These embodiments are provided so that this disclosure will be thorough and complete, and will fully convey the scope of the invention to those skilled in the art. It is to be acknowledged that these embodiments are exemplary implementations and are not to be construed as limiting the scope of the present invention in any way. Further modifications to the disclosed embodiments, as well as other embodiments, are also included within the scope of the appended claims.

These embodiments are provided so that this disclosure is thorough and complete, and fully conveys the inventive concept to those skilled in the art. Regarding the drawings, the relative proportions and ratios of elements in the drawings may be exaggerated or diminished in size for the sake of clarity and convenience. Such arbitrary proportions are only illustrative and not limiting in any way. The same reference numbers are used in the drawings and description to refer to the same or like parts. As used herein, the singular forms “a”, “an” and “the” are intended to include the plural forms as well, unless the context clearly indicates otherwise. As used herein, the term “or” includes any and all combinations of one or more of the associated listed items.

It will be acknowledged that when an element or layer is referred to as being “on,” “connected to” or “coupled to” another element or layer, it can be directly on, connected or coupled to the other element or layer, or intervening elements or layers may be present. In contrast, when an element is referred to as being “directly on,” “directly connected to” or “directly coupled to” another element or layer, there are no intervening elements or layers present.

In addition, unless explicitly described to the contrary, the words “comprise” and “include”, and variations such as “comprises”, “comprising”, “includes”, or “including”, will be acknowledged to imply the inclusion of stated elements but not the exclusion of any other elements.

Before illustration of a surgical decision support system based on augmented reality and a method thereof of the present invention, the environment where the present invention is applied is described first. The present invention is applied through augmented reality, the augmented reality is a technology of using a camera device to capture an image, performing calculation based on positions and an angle of the image, and using image analysis technology to display virtual objects and real-world scenario on a display device and providing a user to interact with the virtual objects. In actual implementation, the display device can be a head-mounted display, a head-up display, or a touch screen.

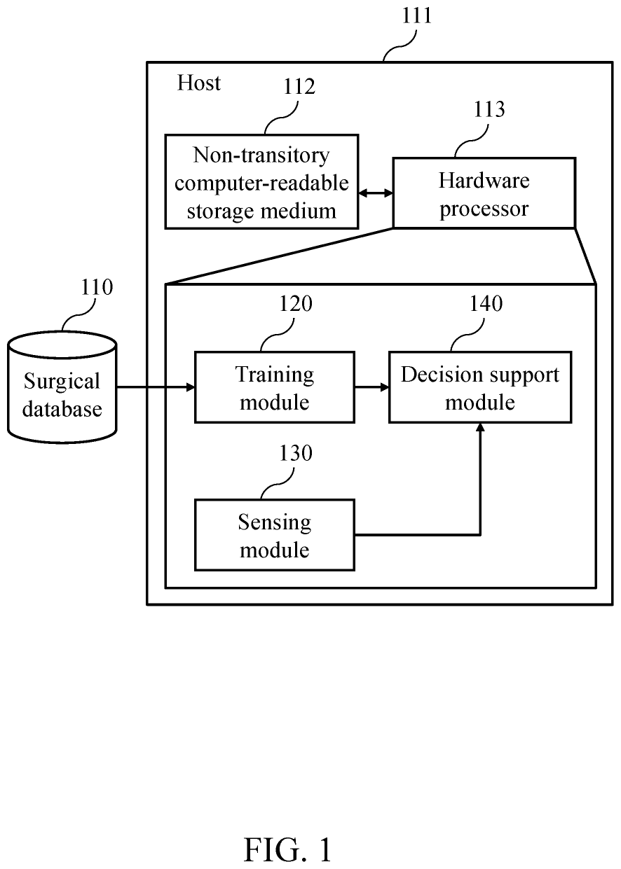

The surgical decision support system based on augmented reality and a method of the present invention will hereinafter be described in more detail with reference to the accompanying drawings in the following paragraphs. Please refer to FIG. 1, FIG. 1 is a system block diagram of a surgical decision support system based on augmented reality, according to the present invention. As shown in FIG. 1, the surgical decision support system includes a surgical database 110 and a host 111. The surgical database 110 is configured to store surgical plans, each of the surgical plans includes an organ model, an operational process, a time point of using surgical instrument, and physiological data, each of the surgical plans is presentable through augmented reality. For example, in a condition that a surgical plan, which is also called surgery method, is hysteron myomectomy, the surgical plan can include organ models of the uterus and its surrounding organs, operational process recording the steps of performing the surgical operation, time points of using surgical instruments that records the various surgical instruments that need to be used in this operation and its use time points, and the physiological data that needs to be paid attention to in performing this operation. In an embodiment, the surgical database 110 can be disposed outside the host 111 or directly disposed in the host 111.

The host 111 is linked with the surgical database 110, and includes a non-transitory computer-readable storage medium 112 and a hardware processor 113. The non-transitory computer-readable storage medium 112 is configured to store computer-readable program instructions, the hardware processor 113 is electrically connected to the non-transitory computer-readable storage medium 112 and configured to execute the computer-readable program instructions to execute a training module 120, a sensing module 130, and a decision support module 140. The training module 120 is linked with the surgical database 110; before a surgery is performed, the training module 120 selects and loads one of surgical plans that corresponds to the surgery for training, and presents the loaded surgical plan through augmented reality; during the training, the training module 120 enables multiple sensors to continuously sense a free motion of a surgical instrument in a three-dimensional space to create an optimal surgical operation. In other words, the surgeon creates the optimal surgical operation during the training process as a comparison basis, it means that the optimal surgical operation of the present invention is not the conventional operation based on textbooks or the operations of famous teachers, so that the created optimal surgical operation is the operation within the surgeon's own ability, to prevent inexperienced surgeons from being unable to reproduce other's surgical operation. In addition, in actual implementation, during the training, the continuously sensed free motion of the surgical instrument can be inputted into the machine learning model as training data, so as to train the machine learning model corresponding to the surgery.

The sensing module 130 is configured to enable the sensor to continuously sense the free motion of the surgical instrument in three-dimensional space to generate a current surgical operation during a surgery process. In actual implementation, when the training module 120 completes the training of the machine learning model, the continuously sensed free motion of the surgical instrument in three-dimensional space can be inputted into the machine learning model during the surgery process, to recognize whether the current surgical operation is similar to the optimal surgical operation.

The decision support module 140 is connected to the training module 120 and the sensing module 130, and configured to compare the optimal surgical operation and the current surgical operation. When a comparison difference exceeds a tolerable range, the decision support module 140 outputs a difference message to provide a surgical decision support. For example, the optimal surgical operation includes data of a specific organ and a moving path of a surgical instrument, the current surgical operation includes data of a specific organ and a moving path of current surgical instrument; in a condition that the optimal surgical operation and the current surgical operation have the same data of the specific organ (for example, the types, locations and sizes of uterine fibroids are the same), when the moving path of the current surgical instrument deviates from the moving path of the surgical instrument of the optimal surgical operation and the deviation exceeds the tolerable range, the deviation is used as a difference message to be outputted. In addition, when the machine learning model is trained completed, the trained machine learning model can be used to recognize whether the current surgical operation is similar to the optimal surgical operation, so as to dynamically adjust the tolerable range based on a recognition result; for example, when the recognition result indicates that the current surgical operation is similar to the optimal surgical operation but the comparison difference exceeds the tolerable range, the tolerable range is extended; when the recognition result indicates that the current surgical operation is not similar to the optimal surgical operation but the comparison result is within the tolerable range, the tolerable range is reduced, so as to keep consistency between the determination result based on the tolerable range and the determination result based on the machine learning.

It is to be particularly noted that, in actual implementation, the modules of the present invention can be implemented by hardware processor, such as integrated circuit chip, system on chip (SOC), a complex programmable logic device (CPLD), or a field programmable gate array (FPGA). The concept of the present invention can be implemented by a system, a method and/or a computer program. The computer program can include computer-readable storage medium which records computer readable program instructions, and the processor can execute the computer readable program instructions to implement concepts of the present invention. The computer-readable storage medium can be a tangible apparatus for holding and storing the instructions executable of an instruction executing apparatus. Computer-readable storage medium can be, but not limited to electronic storage apparatus, magnetic storage apparatus, optical storage apparatus, electromagnetic storage apparatus, semiconductor storage apparatus, or any appropriate combination thereof. More particularly, the computer-readable storage medium can include a hard disk, an RAM memory, a read-only-memory, a flash memory, an optical disk, a floppy disc or any appropriate combination thereof, but this exemplary list is not an exhaustive list. The computer-readable storage medium is not interpreted as the instantaneous signal such a radio wave or other freely propagating electromagnetic wave, or electromagnetic wave propagated through waveguide, or other transmission medium (such as optical signal transmitted through fiber cable), or electric signal transmitted through electric wire. Furthermore, the computer readable program instruction can be downloaded from the computer-readable storage medium to each calculating/processing apparatus, or downloaded through network, such as internet network, local area network, wide area network and/or wireless network, to external computer equipment or external storage apparatus. The network includes copper transmission cable, fiber transmission, wireless transmission, router, firewall, switch, hub and/or gateway. The network card or network interface of each calculating/processing apparatus can receive the computer readable program instructions from network, and forward the computer readable program instruction to store in computer-readable storage medium of each calculating/processing apparatus. The computer program instructions for executing the operation of the present invention can include source code or object code programmed by assembly language instructions, instruction-set-structure instructions, machine instructions, machine-related instructions, micro instructions, firmware instructions or any combination of one or more programming language. The programming language include object oriented programming language, such as Common Lisp, Python, C++, Objective-C, Smalltalk, Delphi, Java, Swift, C #, Perl, Ruby, and PHP, or regular procedural programming language such as C language or similar programming language. The computer readable program instruction can be fully or partially executed in a computer, or executed as independent software, or partially executed in the client-end computer and partially executed in a remote computer, or fully executed in a remote computer or a server.

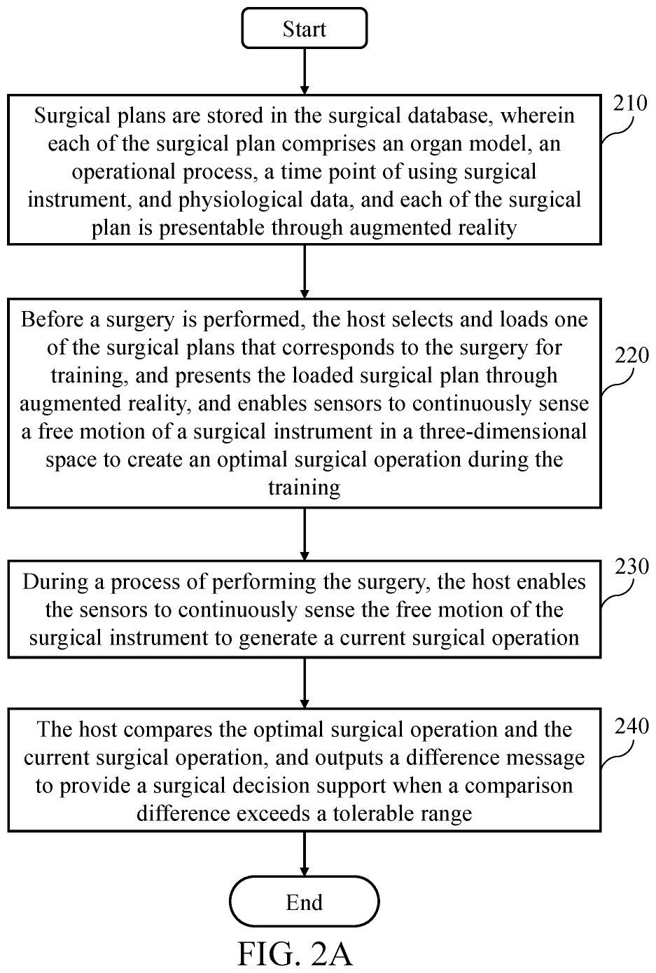

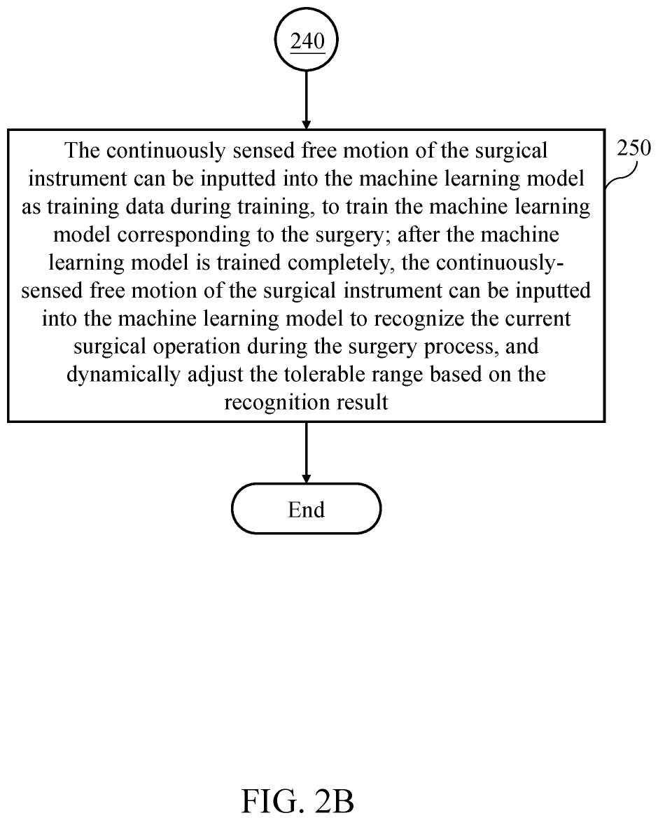

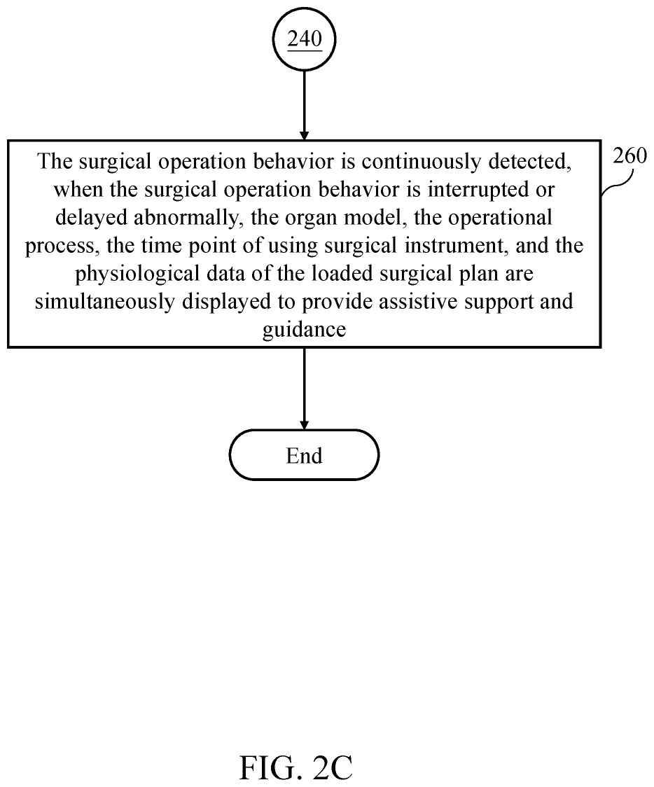

Please refer to FIG. 2A to FIG. 2C, FIG. 2A to FIG. 2C are flowcharts of a surgical decision support method based on augmented reality, according to the present invention. The surgical decision support method is executed by a host 111 linked with a surgical database 110, and includes the following steps. In a step 210, surgical plans are stored in the surgical database 110, wherein each of the surgical plan comprises an organ model, an operational process, a time point of using surgical instrument, and physiological data, and each of the surgical plan is presentable through augmented reality. In a step 220, before a surgery is performed, the host 111 selects and loads one of the surgical plans that corresponds to the surgery for training, and presents the loaded surgical plan through augmented reality, and enables sensors to continuously sense a free motion of a surgical instrument in a three-dimensional space to create an optimal surgical operation during the training. In a step 230, during a process of performing the surgery, the host 111 enables the sensors to continuously sense the free motion of the surgical instrument to generate a current surgical operation. In a step 240, the host 111 compares the optimal surgical operation and the current surgical operation, and outputs a difference message to provide a surgical decision support when a comparison difference exceeds a tolerable range. Through the above-mentioned steps, a surgeon can create the optimal surgical operation before a surgical operation; during the surgical operation, the optimal surgical operation can be demonstrated through augmented reality, the current surgical operation is detected and compared with the optimal surgical operation, the difference message is displayed to provide the surgical decision support when the comparison difference exceeds the tolerable range.

In addition, as shown in FIG. 2B, after the step 240, the continuously sensed free motion of the surgical instrument can be inputted into the machine learning model as training data during training, to train the machine learning model corresponding to the surgery; after the machine learning model is trained completely, the continuously-sensed free motion of the surgical instrument can be inputted into the machine learning model to recognize the current surgical operation during the surgery process, and dynamically adjust the tolerable range based on the recognition result (step 250). In addition, as shown in FIG. 2B, after the step 240, the surgical operation behavior is continuously detected, when the surgical operation behavior is interrupted or delayed abnormally, the organ model, the operational process, the time point of using surgical instrument, and the physiological data of the loaded surgical plan are simultaneously displayed to provide assistive support and guidance (step 260). It is to be noted that, in a condition that the abnormally-interrupted-or-delayed behavior is detected at 6 minutes after the beginning of the operation, at this time, the operational process, the time point of using surgical instrument and the physiological data of the loaded surgical plan after this time point (such as 6 minutes) are displayed simultaneously. Only the part of the surgical plan that has not been performed needs to be displayed, instead of displaying the complete surgical plan from beginning to end.

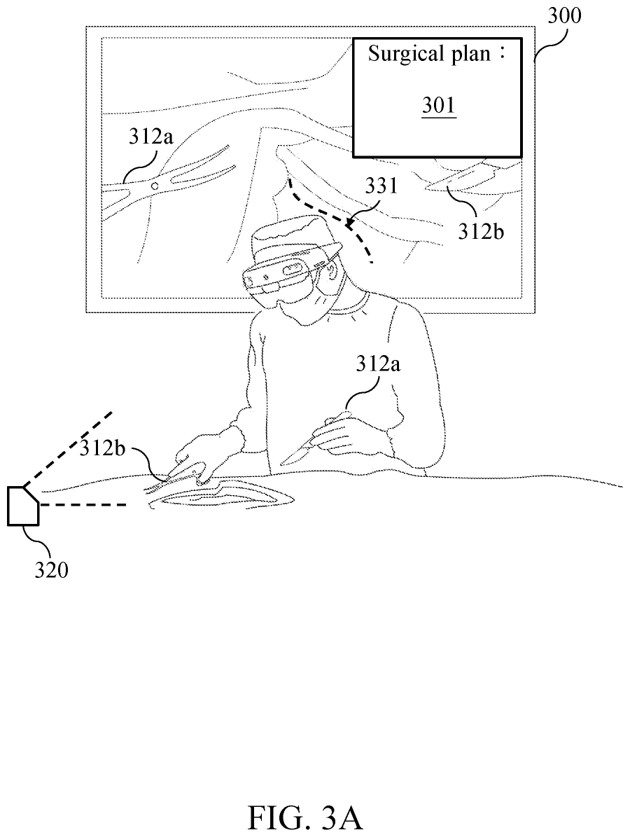

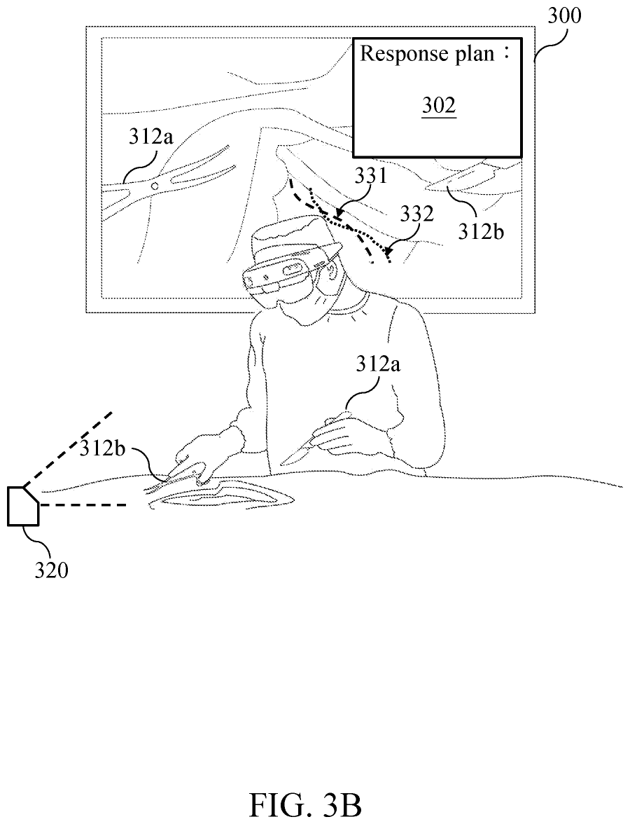

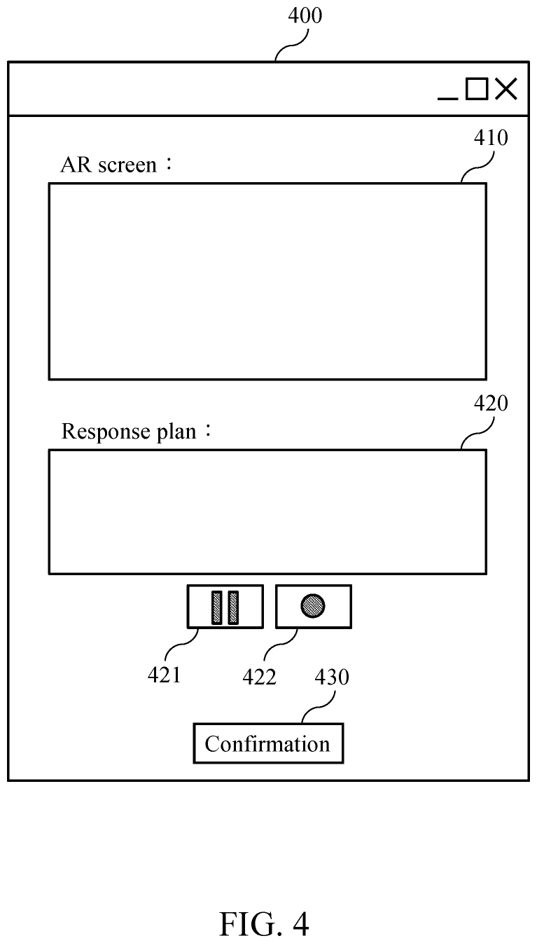

The embodiment of the present invention will be illustrated with reference to FIG. 3A to FIG. 4 in the following paragraphs. As shown in FIG. 3A and FIG. 3B, FIG. 3A and FIG. 3B are schematic views of providing decision supports in a surgical operation at different times, according to an application of the present invention. Before a surgery is performed, a surgeon can load the surgical plan corresponding to the surgery from the surgical database, as shown in FIG. 3A, and the loaded surgical plan is displayed on a display block 301 of the display interface 300, and the images of the organ and tissue corresponding to the surgical plan are also displayed on the display interface, so that the surgeon can perform preoperative training according to the surgical plan. During preoperative training, the free motions of the surgical instruments 312a, 312b in a three-dimensional space are continuously detected through an image sensing technology of the display interface 300 or a motion sensor 320 disposed on the operating table, and recorded as the surgical operation. After multiple preoperative trainings under augmented reality are completely, the surgeon can select the most satisfactory surgical operation as the optimal surgical operation, for example, the movement of the surgical instrument 312b along a dashed line 331 is regarded as the optimal surgical operation. In actual implementation, the surgical plan includes response plans of various emergencies at different time points, such as emergency surgical operations. As shown in FIG. 2B, in the actual operation, the free motions of surgical instruments 312a, 312b can be continuously detected through the motion sensor 320 and recorded as the current surgical operation, for example, the movement of the surgical instrument 312b along a dashed line 332 is used as the current surgical operation. Next, the current surgical operation is compared with the optimal surgical operation; when the difference between the current surgical operation and the optimal surgical operation exceeds the tolerable range, it indicates that the operation process is not smooth from this time point, so that various response plans available for surgeons at this time point to make decisions are displayed on a display block 302, to make the surgeon calmly deal with emergencies. For example, in the middle of the operation, when it is detected that the difference between the moving path of the surgical instrument 312b during training (such as a dashed line 331) and the actual operation (such as a dashed line 332) exceeds the tolerable range, the display block 302 can display the conditions that may occur in the middle of the operation and the corresponding response plans, for the surgeon to select an appropriate response plan, and then the surgical operation of the selected response plan is displayed on the display interface 300 through augmented reality.

Please refer to FIG. 4, FIG. 4 is a schematic view of setting a response plan, according to the present invention. In actual implementation, the response plan can be set through a remote device, for example, an experienced surgeon located at a remote end can open a setting window 400 in the remote device to set the response plan. For example, the remote device receives an AR screen image during training or current operation, and displays the AR screen image on the display block 410, when an inexperienced surgeon encounters an emergency during training or surgery operation, the experienced surgeon at the remote end can type texts in an input block 420, or directly write and draw in the input block 420 with a stylus, and even click a pause component 421 and a recording component 422 to control speech recording, so as to use text, image, and voice as the corresponding response plan; next, experienced surgeon can click a confirmation component 430 to send and store this response plan to the surgical database 110. In this way, when an inexperienced surgeon encounters emergence, the response plan corresponding to this time point can be outputted through the display and the speaker, so that the inexperienced surgeon can calmly deal with emergence.

According to above-mentioned contents, the difference between the present invention and the conventional technology is that, in the present invention, a surgeon can create the optimal surgical operation before a surgical operation; during the surgical operation, the optimal surgical operation can be demonstrated through augmented reality, the current surgical operation is detected and compared with the optimal surgical operation, the difference message is displayed to provide the surgical decision support when the comparison difference exceeds the tolerable range. Therefore, the technical solution of the present invention is able to solve the conventional problem, to achieve the technical effect of improving operation efficiency and success rate.

The present invention disclosed herein has been described by means of specific embodiments. However, numerous modifications, variations and enhancements can be made thereto by those skilled in the art without departing from the spirit and scope of the disclosure set forth in the claims.

What is claimed is:

1. A surgical decision support system based on augmented reality (AR), comprising:
a surgical database, configured to store surgical plans, wherein each of the surgical plans comprises an organ model, an operational process, a time point of using surgical instrument, and physiological data, each of the surgical plans is presentable through augmented reality; and
a host, linked with the surgical database, and comprising:
a non-transitory computer-readable storage medium, configured to store computer-readable program instructions; and
a hardware processor, electrically connected to the non-transitory computer-readable storage medium, and configured to execute the computer-readable program instructions to execute:
a training module, linked with the surgical database, wherein before a surgery is performed, the training module is configured to select and load a corresponding one of the surgical plans for training, present the loaded surgical plan in augmented reality, enable sensors to continuously sense a free motion of a surgical instrument in a three-dimensional space during training, so as to create an optimal surgical operation;
a sensing module, wherein during a process of performing the surgery, the sensing module is configured to enable the sensors to continuously sense the free motion of the surgical instrument in three-dimensional space to generate a current surgical operation; and
a decision support module, connected to the training module and the sensing module, configured to compare the optimal surgical operation and the current surgical operation, wherein when a comparison difference exceeds a tolerable range, the decision support module outputs a difference message to provide a surgical decision support.

2. The surgical decision support system based on augmented reality according to claim 1, wherein the training module inputs the free motion of the surgical instrument that is continuously sensed during the training into a machine learning model as training data, to train the machine learning model corresponding to the surgery, and after the machine learning model is trained completely, the free motion of the surgical instrument that is continuously sensed by the sensing module is permitted to input into the machine learning model to recognize the current surgical operation, and the tolerable range is dynamically adjusted based on a recognition result.

3. The surgical decision support system based on augmented reality according to claim 1, wherein each of the optimal surgical operation and the current surgical operation comprises a sequence of operation steps and ranges of a moving path of the surgical instrument at different time points, wherein when the decision support module detects that at least one of a difference between the sequences of operation steps of the optimal surgical operation and the current surgical operation and a difference between the ranges of the moving paths of the optimal surgical operation and the current surgical operation at the same time point exceeds the tolerable range, the decision support module marks the difference in a significant manner and embeds the difference into the difference message.

4. The surgical decision support system based on augmented reality according to claim 1, wherein each of the surgical plans comprises at least one response plan at different time point, the decision support module loads the response plan corresponding the time point from the surgical database and output the response plan when outputting the difference message, wherein the at least one response plan is permitted to be created by a remote device, and the at least one response plan comprises a text, a voice and an image, and is outputted through a display and a speaker.

5. The surgical decision support system based on augmented reality according to claim 1, wherein the decision support module continuously detects a surgical operation behavior, when the surgical operation behavior is interrupted or delayed abnormally, the decision support module simultaneously displays the organ model, the operational process, the time point of using surgical instrument and the physiological data of the loaded surgical plan, to provide assistive support and guidance.

6. A surgical decision support method based on augmented reality (AR), wherein the surgical decision support method is executed by a host linked with a surgical database, and comprises:
storing surgical plans in the surgical database, wherein each of the surgical plan comprises an organ model, an operational process, a time point of using surgical instrument, and physiological data, and each of the surgical plan is presentable through augmented reality;
before a surgery is performed, selecting and loading one of the surgical plans that corresponds to the surgery for training, and presenting the loaded surgical plan through augmented reality, enabling sensors to continuously sense a free motion of a surgical instrument in a three-dimensional space to create an optimal surgical operation during the training, by the host;
during a process of performing the surgery, enabling the sensors to continuously sense the free motion of the surgical instrument to generate a current surgical operation, by the host; and
comparing the optimal surgical operation and the current surgical operation, and outputting a difference message to provide a surgical decision support when a comparison difference exceeds a tolerable range, by the host.

7. The surgical decision support method based on augmented reality according to claim 6, further comprising:
inputting the free motion of the surgical instrument that is continuously sensed during the training into a machine learning model as training data, to train the machine learning model corresponding to the surgery; and
after the machine learning model is trained completely, permitting the free motion of the surgical instrument that is continuously sensed by the sensing module to input into the machine learning model to recognize the current surgical operation, to dynamically adjust the tolerable range based on a recognition result, by the host.

8. The surgical decision support method based on augmented reality according to claim 6, wherein each of the optimal surgical operation and the current surgical operation comprises a sequence of operation steps and ranges of a moving path of the surgical instrument at different time points, wherein when the decision support module detects that at least one of a difference between the sequences of operation steps of the optimal surgical operation and the current surgical operation and a difference between the ranges of the moving paths of the optimal surgical operation and the current surgical operation at the same time point exceeds the tolerable range, the decision support module marks the difference in a significant manner and embeds the difference into the difference message.

9. The surgical decision support method based on augmented reality according to claim 6, wherein each of the surgical plans comprises at least one response plan at different time point, the decision support module loads the response plan corresponding the time point from the surgical database and output the response plan when outputting the difference message, wherein the at least one response plan is permitted to be created by a remote device, and the at least one response plan comprises a text, a voice and an image, and is outputted through a display and a speaker.

10. The surgical decision support method based on augmented reality according to claim 6, further comprising:
continuously detecting a surgical operation behavior, by the host; and
when the surgical operation behavior is interrupted or delayed abnormally, simultaneously displaying the organ model, the operational process, the time point of using surgical instrument and the physiological data of the loaded surgical plan, to provide assistive support and guidance, by the host.
